(19) United States
(12) Patent Application Publication
(10) Pub. No.: US 2019/0134060 A1
(43) Pub. Date: May 9, 2019

(54) 25-HYDROXYCHOLESTEROL AND METHODS OF USE THEREOF

(71) Applicants: Steve Mennerick, St. Louis, MO (US); Amanda Ann Taylor, St. Louis, MO (US); Min-Yu Sun, St. Louis, MO (US); Charles F. Zorumski, St. Louis, MO (US)

(72) Inventors: Steve Mennerick, St. Louis, MO (US); Amanda Ann Taylor, St. Louis, MO (US); Min-Yu Sun, St. Louis, MO (US); Charles F. Zorumski, St. Louis, MO (US)

(73) Assignee: Washington University, St. Louis, MO (US)

(21) Appl. No.: 16/096,630

(22) PCT Filed: Apr. 25, 2017
(86) PCT No.: PCT/US17/29388

§ 371 Date: Oct. 25, 2018

Related U.S. Application Data
(60) Provisional application No. 62/327,070, filed on Apr. 25, 2016.

(51) Int. Cl. A61K 31/575; A61P 25/00

(52) U.S. Cl. CPC A61K 31/575; A61P 25/00

(57) ABSTRACT

The present disclosure provides compositions comprising 25-hydroxycholesterol, derivatives and prodrugs thereof, and methods of use thereof. Specifically, disclosed herein are methods of decreasing neuronal cell death.

### GOVERNMENTAL RIGHTS

This invention was made with government support under R01 MH101874 awarded by the National Institutes of Health. The government has certain rights in the invention.

### FIELD OF THE INVENTION

The present disclosure provides compositions comprising 25-hydroxycholesterol, derivatives, and prodrugs thereof, and methods of use thereof. Specifically, disclosed herein are methods of decreasing neuronal cell death.

### BACKGROUND OF THE INVENTION

Ischemic stroke is a major cause of death and life disability in the United States, and excitotoxicity is a major outcome of the bioenergetic failure associated with stroke. Energy disruption triggers depolarization, which initiates a positive-feedback cycle of release of glutamate, an important excitatory (depolarizing) transmitter, and subsequent overactivation of N-methyl-D-aspartate receptors (NMDARs), a major subtype of glutamate receptors mediating excitatory transmission throughout the CNS. NMDARs participate in ischemia-induced neuronal injury and death by admitting Ca²⁺ and promoting excitotoxic cell death. Thus, there is a need in the art for a way to protect neuronal cells from death and treat ischemic stroke.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A, FIG. 1B, FIG. 1C, FIG. 1D, and FIG. 1E depict images and graphs showing 24S-HC exacerbates OGD-induced, NMDAR-dependent cell death (FIG. 1A) Representative images showing propidium iodide (PI)-labeled, compromised cells in control, OGD, OGD+24S-HC, and OGD+24S-HC+APV. (FIG. 1B) Survival rate was calculated as number of dead cells normalized to total cells and compared between control, OGD, OGD+APV, OGD+24S-HC, and OGD+24S-HC+APV. Cells treated with OGD and 24S-HC exhibited significantly lower survival rate compared to those treated with OGD alone (n=7; one-way repeated measures ANOVA with Bonferroni's post hoc test, p>0.05). (FIG. 1C) 24S-HC levels in the culture media were measured from control (no virus infection), AAV8-GFP, and AAV8-CYP46A1-GFP infected cell cultures (n=4 cultures for each group; one-way repeated measures ANOVA and Bonferroni post hoc test, *P<0.05). (FIG. 1D) Representative images showing PI-labeled dead cells in GFP-expressing cells and CYP46A1 GFP-expressing cells with or without OGD challenges. (FIG. 1E) Survival rate was compared between control GFP and CYP46A1 GFP-expressing cells with/without OGD, and with OGD+APV. Following OGD treatment, CYP46A1 GFP-expressing cells had significantly lower survival rate compared to GFP-expressing cells (n=5; two-way repeated measures ANOVA with paired t test, *p<0.05).

FIG. 2A, FIG. 2B and FIG. 2C depict graphs showing endogenous 24S-HC exacerbates OGD-induced, NMDAR-dependent cell death. (FIG. 2A) Survival rate was compared between WT and KO cells with/without OGD challenges, and with OGD/APV co-treatment. Following OGD treatment, KO cells had significantly higher survival rate compared to WT cells (n=5 WT and 5 KO; one-way ANOVA with Bonferroni's post hoc test, *p<0.05). (FIG. 2B) 24S-HC concentration (ng/ml) was measured in MEM and the original conditioned medium (CM) immediately following OGD insult and one hour after, respectively. (FIG. 2C) 24S-HC levels measured both immediately following OGD insult and one hour after insult were normalized to 24S-HC measured in the sibling untreated control cultures (n=5 for control cultures and 6 for OGD cultures; P>0.2).

FIG. 3A and FIG. 3B depicts graphs showing that the 24S-HC exacerbation of OGD damage is concentration dependent and can be partially rescued by 25-HC. (FIG. 3A) Survival rate was compared between control, OGD, OGD+50 nM 24S-HC, OGD+0.5 μM 24S-HC, OGD+2 μM 24S-HC, and OGD+2 μM 24S-HC+10 μM 25-HC. Cells treated with OGD and 24S-HC at 0.5 μM and 2 μM showed significantly poorer survival compared to those treated with OGD alone. Application of 25-HC partially prevented OGD-induced cell death exacerbated by 2 μM 24S-HC. (n=11; one-way repeated measures ANOVA with Bonferroni's post hoc test, *p<0.05; **p<0.01; ***P<0.001). (FIG. 3B) 25-HC not only partially rescued OGD-induced cell death following SGE-201 application, but also protected against OGD-induced cell death in the absence of 24S-HC analogue application (n=7; one-way repeated measures ANOVA with Bonferroni's post hoc test, *p<0.05; **p<0.01; ***P<0.001).

FIG. 4A and FIG. 4B depict graphs showing that 25-HC alone is neuroprotective against OGD-induced cell death, independent of NMDARs. (FIG. 4A) Survival rates from cultures treated with APV before or after OGD insult were normalized to the survival rate of cultures treated with OGD alone. APV treated before OGD insult has significantly larger rescuing effects on the survival rate compared to APV treated after OGD insult (n=7 for APV-before; n=15 for APV-after; *P<0.05). (FIG. 4B) Survival rates from cultures treated with 25-HC before or after OGD insult were normalized to the survival rate of cultures treated with OGD alone. 25-HC treated before and after OGD insult have indistinguishable rescuing effects on the survival rate (n=15 for each group; P>0.2).

FIG. 5A and FIG. 5B depict graphs showing that 25-HC alone does not protect against NMDA-induced cell death, but it alleviates exacerbation of toxicity by 24S-HC. (FIG. 5A) Application of NMDA at 8 μM and 20 μM significantly reduced cell survival, which was not inhibited when cells were co-treated with 10 μM 25-HC (n=13, one-way repeated measures ANOVA with Bonferroni's post hoc test, ***p<0.001). (FIG. 5B) Application of 24S-HC significantly exacerbated 8 μM NMDA-induced cell death. The exacerbation was inhibited by 10 μM 25-HC (n=6, one-way repeated measures ANOVA with Bonferroni's post hoc test, *p<0.05).

FIG. 6A, FIG. 6B, FIG. 6C, FIG. 6D, and FIG. 6E depict graphs showing that 25-HC has an NMDAR-independent protecting effect on OGD-induced cell death. (FIG. 6A) Survival rate was compared between control, OGD (3 hour), OGD (3 hour)+MK-801, and OGD (3 hour) +MK-801+25-HC. Cell death induced by more severe OGD (3 hour) was only partially inhibited by MK-801 application. 25-HC protected against the MK-801 insensitive OGD-induced cell death. (n=12 cultures for each group, one-way repeated measures ANOVA with Bonferroni's post hoc test, *p<0.05, ***p<0.001). (FIG. 6B) SGE-201 did not increase cell death, verifying the NMDAR independence (N=9 cultures for each group; P>0.05). (FIG. 6C) H₂O₂ (100 μM, 1 hour treatment) toxicity was used to test neuroprotection of 25-HC. 25-HC again yielded mild but reliable protection (N=10 cultures for each group, one-way repeated measures ANOVA with Bonferroni's post hoc test, *P<0.05). (FIG. 6D) and (FIG. 6E) Neither WT nor CYP46A1 knockout hippocampal neuron cultures from C571Bl/6 mice was sensitive to the mild NMDAR independent neuroprotective effect of 25-HC (N=7 cultures for each group; P>0.05).

### DETAILED DESCRIPTION OF THE INVENTION

Provided herein is a method of decreasing neuronal cell death through the administration of 25-hydroxycholesterol, or derivatives, and prodrugs thereof. Importantly, neuroprotection was observed when 25-hydroxycholesterol, or derivatives, and prodrugs thereof, was administered both before and after the onset of neuronal cell death. Accordingly, administration of 25-hydroxycholesterol provides an effective strategy to both prevent and treat neuronal cell death.

Various aspects of the disclosure are described in greater detail below.

### I. Composition

In an aspect, the disclosure provides a composition comprising 25-hydroxycholesterol (25-HC). 25-HC may also be referred to as 5-cholestene-3β,25-diol, the structure of which is diagramed below, and has the empirical formula C₂₇H₄₆O₂, with a molecular weight of 402.65 and a CAS number 2140-46-7.

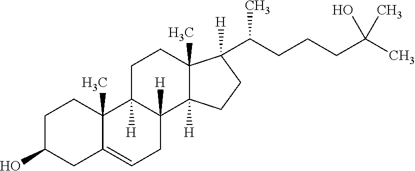

In another aspect, the disclosure provides a composition comprising a derivative of 25-HC. A 25-HC derivative may be modified to improve potency, bioavailability, solubility, stability, handling properties, or a combination thereof, as compared to an unmodified version, provided it has the same or similar activity of 25-HC. In still another aspect, the disclosure provides a composition comprising a prodrug of 25-HC. As used herein, a prodrug is a compound that, after administration, is metabolized (i.e., converted within the body) into a pharmacologically active drug.

A composition of the disclosure may optionally comprise one or more additional drugs or therapeutically active agents in addition to 25-HC, a 25-HC derivative, or a 25-HC prodrug. A composition of the disclosure may further comprise a pharmaceutically acceptable excipient, carrier or diluent. Further, a composition of the invention may contain preserving agents, solubilizing agents, stabilizing agents, wetting agents, emulsifiers, sweeteners, colorants, odorants, salts (substances of the present invention may themselves be provided in the form of a pharmaceutically acceptable salt), buffers, coating agents or antioxidants.

Dosages of 25-HC, a 25-HC derivative, or a 25-HC prodrug can vary between wide limits, depending upon the disease or disorder to be treated, the age of the subject, and the condition of the subject to be treated. In an embodiment where a composition comprising 25-HC, a 25-HC derivative, or a 25-HC prodrug is contacted with a sample, the concentration of 25-HC, a 25-HC derivative, or a 25-HC prodrug may be from about 1 μM to about 50 μM. Alternatively, the concentration of 25-HC, a 25-HC derivative, or a 25-HC prodrug may be from about 5 μM to about 25 μM. For example, the concentration of 25-HC, a 25-HC derivative, or a 25-HC prodrug may be about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 30, about 35, about 40, about 45 or about 50 μM. Additionally, the concentration of the 25-HC, a 25-HC derivative, or a 25-HC prodrug may be greater than 50 μM. For example, the concentration of 25-HC, a 25-HC derivative, or a 25-HC prodrug may be about 50, about 55, about 60, about 65, about 70, about 75, about 80, about 85, about 90, about 95 or about 100 μM.

In an embodiment where the composition comprising 25-HC, a 25-HC derivative, or a 25-HC prodrug is administered to a subject, the dose of 25-HC, a 25-HC derivative, or a 25-HC prodrug may be from about 0.1 mg/kg to about 500 mg/kg. For example, the dose of 25-HC, a 25-HC derivative, or a 25-HC prodrug may be about 0.1 mg/kg, about 0.5 mg/kg, about 1 mg/kg, about 5 mg/kg, about 10 mg/kg, about 15 mg/kg, about 20 mg/kg, or about 25 mg/kg. Alternatively, the dose of 25-HC, a 25-HC derivative, or a 25-HC prodrug may be about 25 mg/kg, about 50 mg/kg, about 75 mg/kg, about 100 mg/kg, about 125 mg/kg, about 150 mg/kg, about 175 mg/kg, about 200 mg/kg, about 225 mg/kg, or about 250 mg/kg. Additionally, the dose of 25-HC, a 25-HC derivative, or a 25-HC prodrug may be about 300 mg/kg, about 325 mg/kg, about 350 mg/kg, about 375 mg/kg, about 400 mg/kg, about 425 mg/kg, about 450 mg/kg, about 475 mg/kg or about 500 mg/kg.

### (a) Components of the Composition

The present disclosure also provides pharmaceutical compositions. The pharmaceutical composition comprises 25-HC, a 25-HC derivative, or a 25-HC prodrug, as an active ingredient, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylam ides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In a further embodiment, the excipient may be a lubricant. Non-limiting examples of suitable lubricants include minerals such as talc or silica; and fats such as vegetable stearin, magnesium stearate or stearic acid.

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The composition can be formulated into various dosage forms and administered by a number of different means that will deliver a therapeutically effective amount of the active ingredient. Such compositions can be administered orally, parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Gennaro, A. R., Remington's Pharmaceutical Sciences, Mack Publishing Co., Easton, Pa. (18^{th} ed, 1995), and Liberman, H. A. and Lachman, L., Eds., Pharmaceutical Dosage Forms, Marcel Dekker Inc., New York, N.Y. (1980). In a specific embodiment, a composition may be a food supplement.

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition comprising 25-HC, a 25-HC derivative, or a 25-HC prodrug is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of 25-HC, a 25-HC derivative, or a 25-HC prodrug in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, a 25-HC, a 25-HC derivative, or a 25-HC prodrug may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9,12,15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying 25-HC, a 25-HC derivative, or a 25-HC prodrug may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In another embodiment, a composition of the invention may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. 25-HC, a 25-HC derivative, or a 25-HC prodrug may be encapsulated in a microemulsion by any method generally known in the art.

In yet another embodiment, 25-HC, a 25-HC derivative, or a 25-HC prodrug may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

### II. Methods

In an aspect, the disclosure provides a method of decreasing neuronal cell death. The method comprises administering a composition comprising 25-hydroxycholesterol (25-HC), a 25-HC derivative, or a 25-HC prodrug.

As used herein, a neuronal cell, may also be referred to as a neuron or a nerve cell, is an electrically excitable cell that processes and transmits information through electrical and chemical signals. Neurons are the core components of the brain and spinal cord of the central nervous system (CNS), and of the ganglia of the peripheral nervous system (PNS). Specialized types of neurons include: sensory neurons which respond to touch, sound, light and all other stimuli affecting the cells of the sensory organs that then send signals to the spinal cord and brain, motor neurons that receive signals from the brain and spinal cord to cause muscle contractions and affect glandular outputs, and interneurons which connect neurons to other neurons within the same region of the brain, or spinal cord in neural networks.

Neuroprotection may be determined by measuring cell death of neuronal cells. Methods of measuring cell death are known in the art. For example, cell death may be measured by Giemsa staining, trypan blue exclusion, acridine orange/ethidium bromide (AO/EB) double staining for fluorescence microscopy and flow cytometry, propidium iodide (PI) staining, annexin V assay, TUNEL assay, DNA ladder, LDH activity, and MTT assay. Cell death may be due to induction of apoptosis. Cell death due to induction of apoptosis may be measured by observation of morphological characteristics including cell shrinkage, cytoplasmic condensation, chromatin segregation and condensation, membrane blebbing, and the formation of membrane-bound apoptotic bodies. Cell death due to induction of apoptosis may be measured by observation of biochemical hallmarks including internucleosomal DNA cleavage into oligonucleosome-length fragments. Traditional cell-based methods of measuring cell death due to induction of apoptosis include light and electron microscopy, vital dyes, and nuclear stains. Biochemical methods include DNA laddering, lactate dehydrogenase enzyme release, and MTT/XTT enzyme activity. Additionally, terminal deoxynucleotidyl transferase-mediated dUTP-biotin nick end labeling of DNA fragments (TUNEL) and in situ end labeling (ISEL) techniques are used, which when used in conjunction with standard flow cytometric staining methods yield informative data relating cell death to various cellular parameters, including cell cycle and cell phenotype. See Loo and Rillema, Methods Cell Biol. 1998; 57:251-64, which is incorporated herein by reference, for a review of these methods.

Neuroprotection may be determined by reducing the signs or symptoms associated with stroke. For example, signs or symptoms of associated with a stroke include trouble with speaking and understanding; paralysis or numbness of the face, arm, or leg; trouble with seeing in one or both eyes; headache(s); trouble with walking; etc.

Neuroprotection may be determined by reducing the signs or symptoms associated with a neurodegenerative disease. For example, signs or symptoms associated with a neurodegenerative disease include memory loss; loss of control in walking, balance, mobility, vision, speech, and swalling; loss of behavior control, emotion, and language; etc.

The results of these methods may be used to determine the percentage of viable cells. In an embodiment, cell death may be measured as a reduction in viable cells. Since a composition of the disclosure decreases neuronal cell death, an increase in viable cells relative to untreated neuronal cells undergoing cell death is indicative of decreasing neuronal cell death. As such, an increase in viable cells following administration of 25-HC, a 25-HC derivative, or a 25-HC prodrug may be greater than 1° A relative to untreated neuronal cells undergoing cell death. For example, an increase in viable cells may be greater than 1%, greater than 2%, greater than 3%, greater than 4%, or greater than 5% relative to untreated neuronal cells undergoing cell death. Alternatively, an increase in viable cells may be greater than 5%, greater than 6%, greater than 7%, greater than 8%, greater than 9%, or greater than 10% relative to untreated neuronal cells undergoing cell death. Additionally, an increase in viable cells may be greater than 10%, greater than 11%, greater than 12%, greater than 13%, greater than 14%, or greater than 15% relative to untreated neuronal cells undergoing cell death. Further, an increase in viable cells may be greater than 15%, greater than 20%, greater than 25%, greater than 30%, greater than 35%, greater than 40%, greater than 45%, or greater than 50% relative to untreated neuronal cells undergoing cell death. Still further, an increase in viable cells may be greater than 50%, greater than 55%, greater than 60%, greater than 65%, greater than 70%, greater than 75%, or greater than 80%, greater than 85%, or greater than 90%, or greater than 95% relative to untreated neuronal cells undergoing cell death.

In another embodiment, an increase in viable cells relative to untreated neuronal cells undergoing cell death is measured using p-value. For instance, when using p-value, an increase in viable cells relative to untreated neuronal cells undergoing cell death following administration of 25-HC, a 25-HC derivative, or a 25-HC prodrug occurs when the p-value is less than 0.1, preferably less than 0.05, more preferably less than 0.01, even more preferably less than 0.005, the most preferably less than 0.001.

In certain embodiments, the neuronal cell death is due to ischemia. In other embodiments, the neuronal cell death is due to stroke.

In still another aspect, the disclosure provides a method of treating or preventing stroke. The method comprises administering a composition comprising 25-HC, a 25-HC derivative, or a 25-HC prodrug. A stroke occurs when the blood supply to part of the brain is interrupted or severely reduced, depriving brain tissue of oxygen and nutrients. A suitable subject may or may not be at risk for a stroke. Non-limiting examples of risk factors for stroke include overweight or obese, physical inactivity, heavy or binge drinking, use of illicit drugs such as cocaine and methamphetamines, high blood pressure, cigarette smoking or exposure to second hand smoke, high cholesterol, diabetes, obstructive sleep apnea, cardiovascular disease including heart failure, heart defects, heart infection or abnormal heart rhythm, personal or family history of stroke, heart attack or transient ischemic attack, 55 or older, race (African Americans have a higher risk), gender (men have a higher risk). A suitable subject may or may not have a sign or symptom associated with stroke. Non-limiting examples of signs or symptoms associated with stroke include trouble speaking and understanding, paralysis or numbness of the face, arm or leg, trouble seeing in one or both eyes, headache, and/or trouble with walking. Specifically, the stroke may be ischemic stroke. Ischemic stroke may be thrombotic stroke or embolic stroke. Additionally, the stroke may be a transient ischemic attack (TIA), also referred to as a ministroke.

In still yet another aspect, the disclosure provides a method of treating or preventing a disease associated with neuronal cell degeneration. In an embodiment, the disclosure provides a method of treating or preventing a neurodegenerative disease. As used herein, a “neurodegenerative disease” is a term for a range of conditions that primarily affect the neurons of the nervous system resulting in degeneration and/or death of nerve cells. Non-limiting examples of neurodegenerative diseases include amyotrophic lateral sclerosis, Parkinson's disease, Alzheimer's disease, Huntington's disease, motor neuron diseases, spinocerebellar ataxia, spinal muscular atrophy, and prion disease. Non-limiting examples of diseases or disorders that may be associated with neuronal cell death or degeneration include schizophrenia, depression, bipolar disorder (Type I or Type II), schizoaffective disorder, mood disorders, anxiety disorders, personality disorders, psychosis, compulsive disorders, post-traumatic stress disorder (PTSD), Autism spectrum disorder (ASD), dysthymia (mild depression), social anxiety disorder, obsessive compulsive disorder (OCD), pain (e.g., a painful syndrome or disorder), sleep disorders, memory disorders (e.g., memory impairment), dementia, Alzheimer's Disease, a seizure disorder (e.g., epilepsy), traumatic brain injury, stroke, addictive disorders (e.g., addiction to opiates, cocaine, and/or alcohol), autism, Huntington's Disease, insomnia, Parkinson's disease, withdrawal syndromes, and tinnitus.

As used herein, the term “preventing” or “prevention” or “prophylactic treatment” refers to a reduction in risk of acquiring or developing a disease or disorder (i.e., causing at least one of the clinical symptoms of the disease not to develop) in a subject. The subject may or may not be predisposed to the disease in advance of disease onset. As used herein, the term “prophylaxis” is related to “prevention,” and refers to a measure to prevent, rather than to treat or cure a disease.

As used herein, the term “treating” or “treatment” or “therapeutic treatment” of any disease or disorder refers to ameliorating the disease or disorder (i.e., arresting the disease or reducing the manifestation, extent or severity of at least one of the clinical symptoms thereof). Additionally, “treating” or “treatment” may refer to ameliorating at least one physical parameter, which may not be discernible by the subject. In another embodiment, “treating” or “treatment” may refer to modulating the disease or disorder, either physically, (e.g., stabilization of a discernible symptom), physiologically, (e.g., stabilization of a physical parameter), or both.

The composition is as described in Section I. The subject and administration are described below.

### (a) Administration

In certain aspects, a pharmacologically effective amount of a composition of the disclosure may be administered to a subject. Administration is performed using standard effective techniques, including peripherally (i.e., not by administration into the central nervous system) or locally to the central nervous system. Peripheral administration includes but is not limited to intravenous, intraperitoneal, subcutaneous, intratumoral, pulmonary, transdermal, intramuscular, intranasal, buccal, sublingual, or suppository administration. Local administration, including directly into the central nervous system (CNS) includes but is not limited to via a lumbar, intraventricular or intraparenchymal catheter or using a surgically implanted controlled release formulation. Pheresis may be used to deliver a composition of the invention. In certain embodiments, a composition of the invention may be administered via an infusion (continuous or bolus).

Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners.

Suitable vehicles for peripheral systemic delivery by intravenous or intraperitoneal or subcutaneous or intramuscular injection are straightforward. In addition, however, administration may also be effected through the mucosal membranes by means of nasal aerosols or suppositories. Suitable formulations for such modes of administration are well known and typically include surfactants that facilitate cross-membrane transfer. Such surfactants are often derived from steroids or are cationic lipids, such as N-[1-(2,3-dioleoyl)propyl]-N,N,N-trimethyl ammonium chloride (DOTMA) or various compounds such as cholesterol hemisuccinate, phosphatidyl glycerols and the like.

For therapeutic applications, a therapeutically effective amount of a composition of the invention is administered to a subject. A “therapeutically effective amount” is an amount of the therapeutic composition sufficient to produce a measurable response (e.g., neuroprotection, reduction in neuronal cell death, increase in the number of neuronal cells, reduction in the signs or symptoms associated with neuronal death, reduction in the signs or symptoms associated with stroke, reduction in the signs or symptoms associated with a neurodegenerative disease). Actual dosage levels of active ingredients in a therapeutic composition of the invention can be varied so as to administer an amount of the active ingredient(s) that is effective to achieve the desired therapeutic response for a particular subject. The selected dosage level will depend upon a variety of factors including the activity of the therapeutic composition, formulation, the route of administration, combination with other drugs or treatments, size and longevity of ischemic episode, and the physical condition and prior medical history of the subject being treated. In some embodiments, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

The frequency of dosing may be once, twice, three times or more daily or once, twice, three times or more per week or per month, as needed as to effectively treat the symptoms or disease. In certain embodiments, the frequency of dosing may be once, twice or three times daily. For example, a dose may be administered every 24 hours, every 12 hours, or every 8 hours. In a specific embodiment, the frequency of dosing may be twice daily.

Duration of treatment could range from a single dose administered on a one-time basis to a life-long course of therapeutic treatments. The duration of treatment can and will vary depending on the subject and the cancer or autoimmune disease or infection to be treated. For example, the duration of treatment may be for 1 day, 2 days, 3 days, 4 days, 5 days, 6 days, or 7 days. Or, the duration of treatment may be for 1 week, 2 weeks, 3 weeks, 4 weeks, 5 weeks or 6 weeks. Alternatively, the duration of treatment may be for 1 month, 2 months, 3 months, 4 months, 5 months, 6 months, 7 months, 8 months, 9 months, 10 months, 11 months, or 12 months. In still another embodiment, the duration of treatment may be for 1 year, 2 years, 3 years, 4 years, 5 years, or greater than 5 years. It is also contemplated that administration may be frequent for a period of time and then administration may be spaced out for a period of time. For example, duration of treatment may be 5 days, then no treatment for 9 days, then treatment for 5 days.

The timing of administration of the treatment relative to the disease itself and duration of treatment will be determined by the circumstances surrounding the case. Treatment could begin immediately, such as at the time of diagnosis, or treatment could begin following surgery. Treatment could begin in a hospital or clinic itself, or at a later time after discharge from the hospital or after being seen in an outpatient clinic.

Although the foregoing methods appear the most convenient and most appropriate and effective for administration of a composition of the invention, by suitable adaptation, other effective techniques for administration, such as intraventricular administration, transdermal administration and oral administration may be employed provided proper formulation is utilized herein.

In addition, it may be desirable to employ controlled release formulations using biodegradable films and matrices, or osmotic mini-pumps, or delivery systems based on dextran beads, alginate, or collagen.

A composition of the disclosure may be administered before or during oxygen-glucose deprivation (OGD). OGD results when cells (e.g., neuronal cells) are in an environment with a reduced supply of oxygen and glucose.

A composition of the disclosure may also be administered in combination with standard treatment or prevention for stroke. Standard treatment or prevention may depend on the type and severity of stroke, as well as the general condition of the subject. Non-limiting examples of standard treatment or prevention for ischemic stroke include aspirin, tissue plasminogen activator (TPA), mechanical thrombectomy, carotid endarterectomy, angioplasty and stents.

### (b) Subject

A suitable subject may include a human, a livestock animal, a companion animal, a lab animal, or a zoological animal. The subject may be a pediatric subject (e.g., infant, child, adolescent) or an adult subject (e.g., young adult, middle-aged adult or senior adult). In one embodiment, the subject may be a rodent, e.g., a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a specific embodiment, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In preferred embodiments, the subject is a human.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Introduction to the Examples

Ischemic stroke is a major cause of death and life disability in the United States, and excitotoxicity is a major outcome of the bioenergetic failure associated with stroke (1). Energy disruption triggers depolarization, which initiates a positive-feedback cycle of release of glutamate, an important excitatory (depolarizing) transmitter, and subsequent overactivation of N-methyl-D-aspartate receptors (NMDARs), a major subtype of glutamate receptors mediating excitatory transmission throughout the CNS. NMDARs participate in ischemic-induced neuronal injury and death by admitting Ca²⁺ and promoting excitotoxic cell death (2). Therefore, diminishing NMDAR-mediated excitotoxicity may be neuroprotective and benefit stroke outcome. However, undesired side effects have limited the strategy of directly inhibiting/blocking NMDARs as therapy. Understanding and targeting endogenous positive allosteric modulators of NMDAR function my produce fewer downsides since basal NMDAR function remains unaltered.

24S-hydroxycholesterol (24S-HC) and its synthetic analogues are a novel class of positive NMDAR modulators. 24S-HC is the major brain cholesterol metabolite. It is produced by cholesterol 24-hydroxylase (CYP46A1), a neuron-specific enzyme localized to dendrites (3). It is found abundantly in adult brain tissue (30-60 μg/g tissue), suggesting that it may regulate normal and pathophysiological NMDAR activity (4, 5).

Herein, the contributions of 24S-HC to neuronal death associated with oxygen-glucose deprivation (OGD) are assessed, especially the role of NMDARs in the actions of 24S-HC. Primary hippocampal neurons are exploited, where 24S-HC levels can be readily controlled and measured. It was explored whether manipulating exogenous or endogenous levels of 24S-HC is neuroprotective against OGD-induced excitotoxicity. The concentrations of 24S-HC that exacerbate in vitro injury caused by OGD were examined, and it was explored whether exacerbation by 24S-HC can be entirely ascribed to NMDAR modulation. Moreover, it was investigated whether 25-hydroxycholesterol (25-HC), an oxysterol, has beneficial effects against OGD-induced neuronal death. It was found that 25-HC neuroprotection is partly dependent on its antagonism of 24S-HC but surprisingly also includes a component that is independent of NMDARs. In sum, targeting an endogenous positive allosteric modulator of NMDARS or targeting the novel NMDAR independent mechanism described here may avoid potentially deleterious side effects of NMDAR antagonists as therapeutics (6).

### Example 1

### 24S-HC Exacerbates OGD Damage, which is APV Sensitive

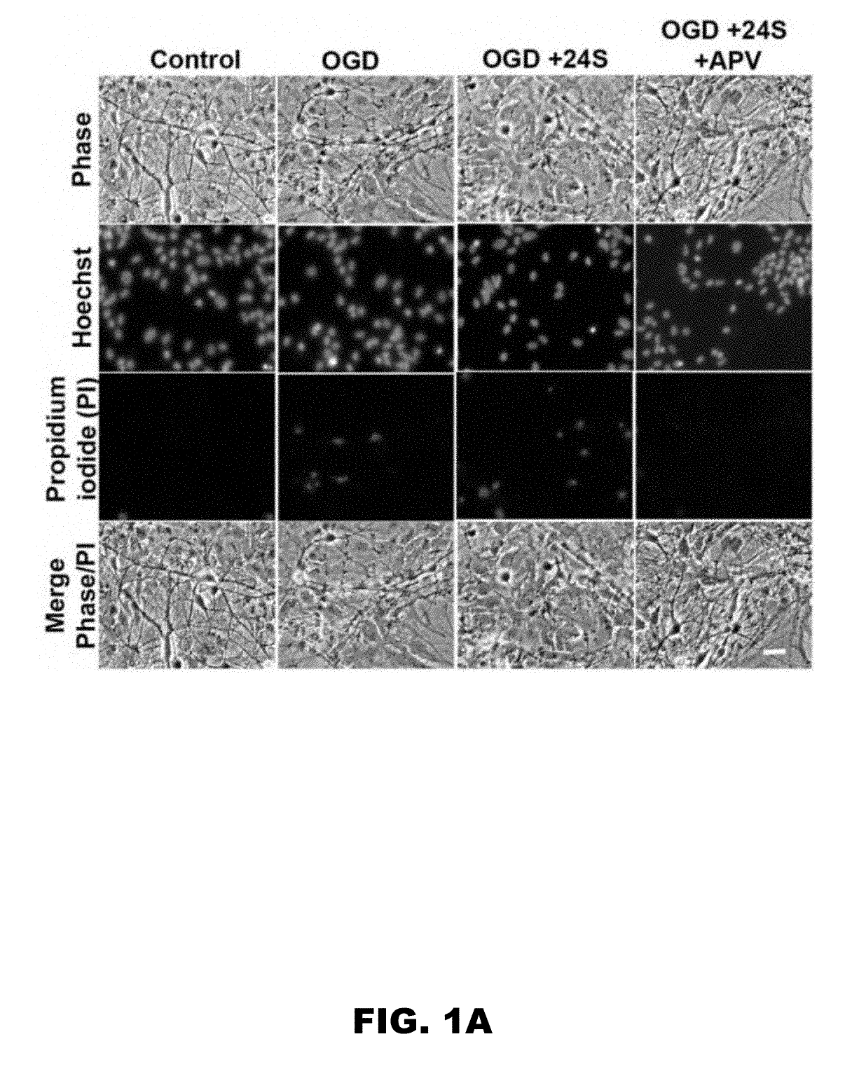

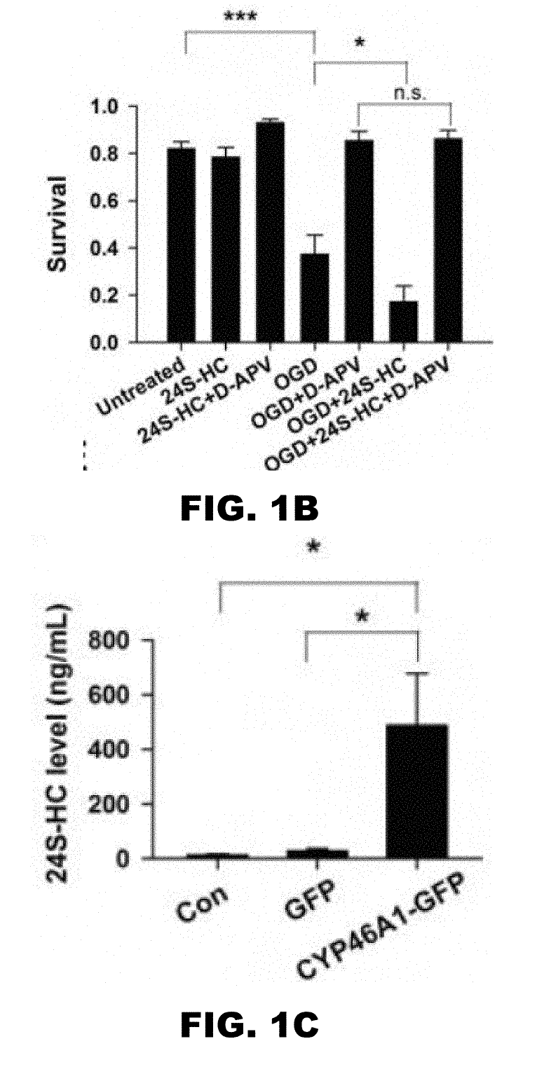

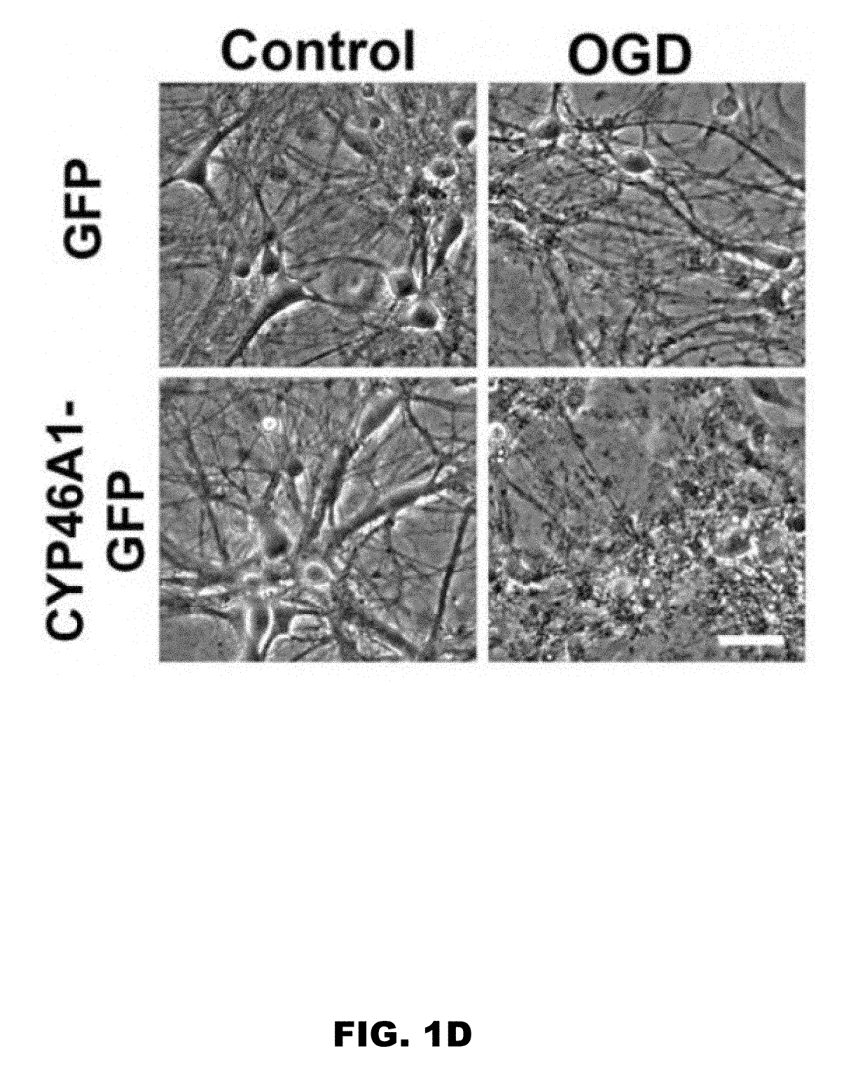

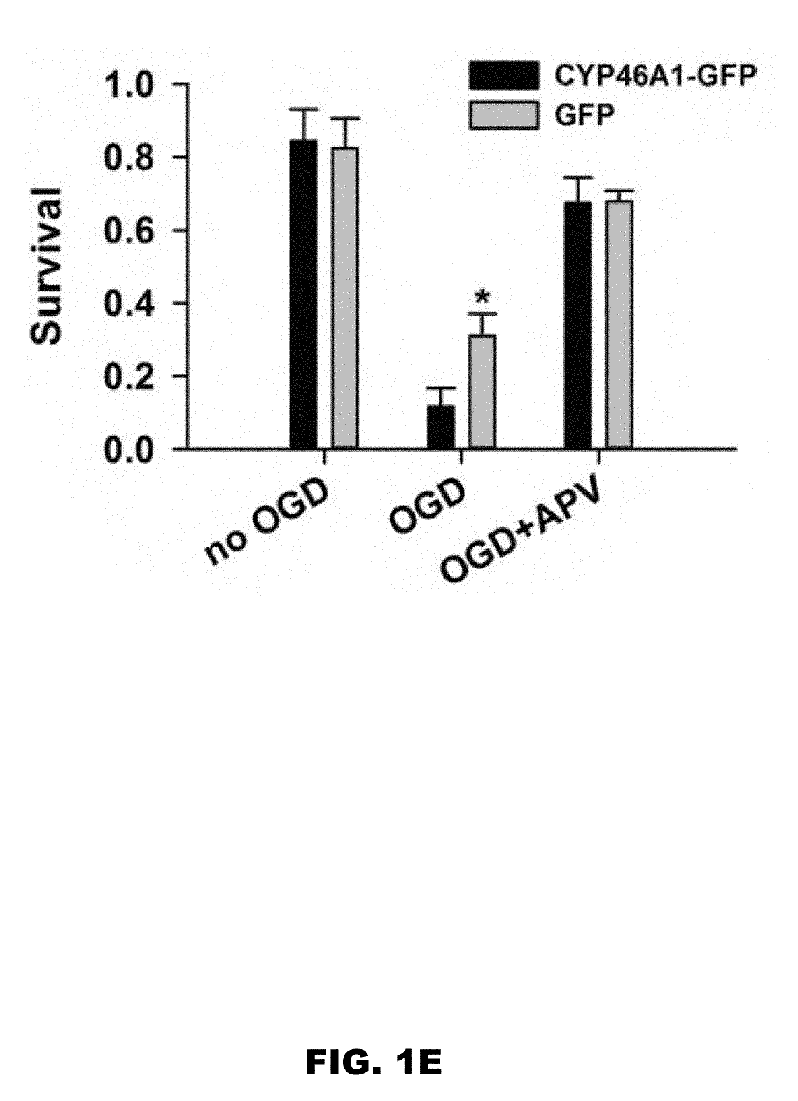

Cell death induced by hypoxia and OGD in hippocampal cultures is NMDAR dependent (7-10). Because 24S-HC and its analogues increase NMDAR activity (11), these compounds may exacerbate OGD-induced cell death by potentiating NMDAR activity. Effects of 24S-HC on NMDAR function saturate at ˜10 μM (11). Exogenous application of 24S-HC at 2 μM to WT rat hippocampal cultures 14 days in vitro enhanced OGD-induced cell death (FIG. 1A and FIG. 1B). This cell death was rescued by co-treatment with an NMDAR antagonist, APV, prior to and during OGD, confirming that the exacerbation of cell death was NMDAR dependent.

It was tested whether elevation of 24S-HC by genetically overexpressing CYP46A1 to mimic mature 24S-HC levels could augment endogenous 24S-HC and OGD-induced toxicity. WT rat primary hippocampal cultures were infected with an AAV-CYP-GFP virus. Conditioned culture medium from cells infected with AAV-CYP46A1-GFP exhibited significant elevation of 24S-HC level compared to control AAV-GFP infected cultures (FIG. 1C), verifying virus effectiveness. Overexpression of CYP46A1 increased cell death following OGD relative to controls (FIG. 1D and FIG. 1E). Again, APV prevented OGD-induced cell death in both AAV-CYP-GFP and AAV-GFP-infected neuron cultures (FIG. 1E), implicating NMDARs in the increased damage. Taken together, these results suggest that 24S-HC exacerbates OGD-induced damage, and its actions exclusively involve NMDAR activation. Furthermore, because OGD occurs in unconditioned culture medium devoid of 24S-HC (see Methods and below), the results suggest that locally elevated levels of 24S-HC may drive NMDAR activity to exacerbate damage.

### Example 2

### Endogenous 24S-HC Exacerbates OGD-Induced Damage

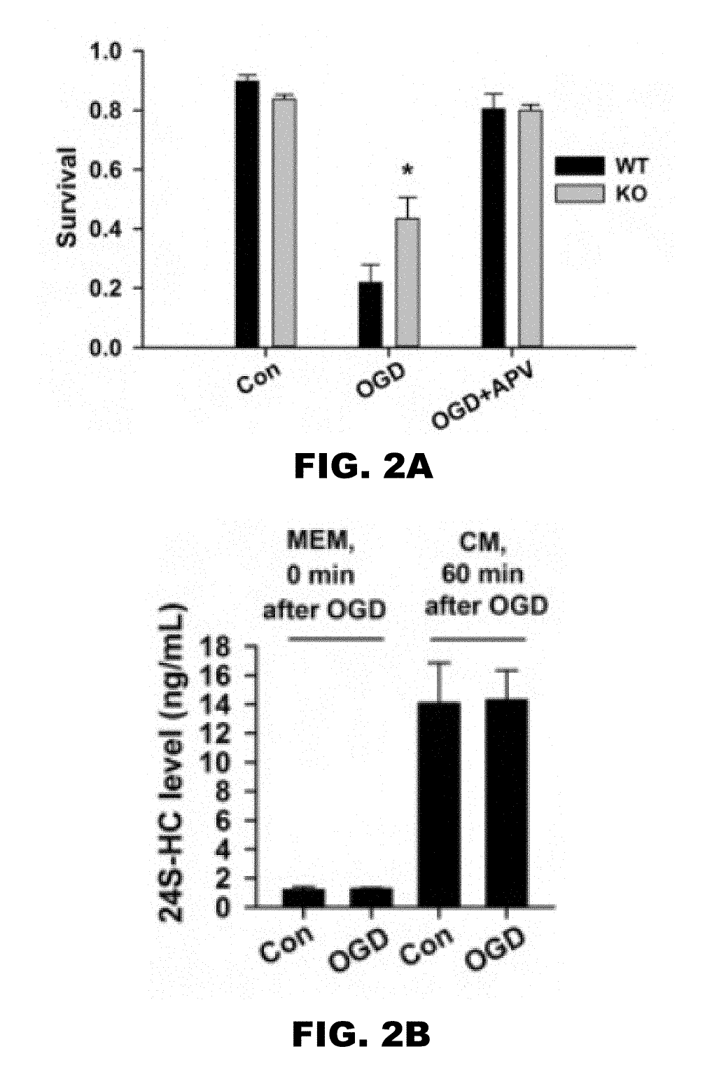

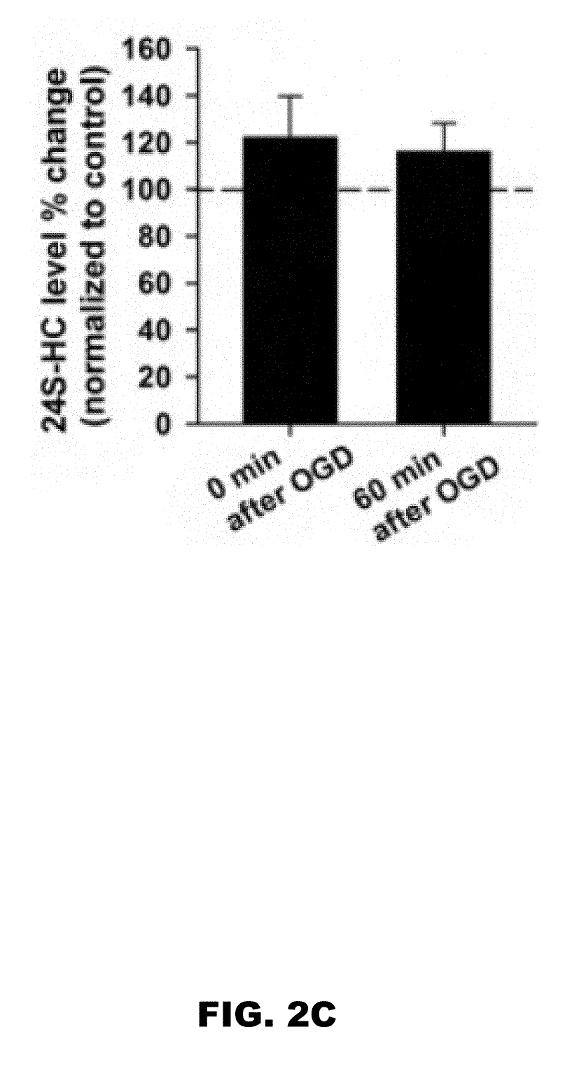

It was next investigated whether down-regulation of endogenous 24S-HC protects against OGD-induced cell death. In CYP46A1 knockout (KO) mice, 24S-HC is greatly reduced, and deficits of NMDAR-dependent functions including synaptic plasticity, learning and memory have been reported (3, 8), suggesting reduced NMDAR activity in these mice. When comparing OGD-induced cell death in WT and KO hippocampal cultures, a significantly higher survival rate in KO cultures was observed, suggesting that reduction of endogenous 24S-HC protects against OGD-induced damage (FIG. 2).

Because basal levels of 24S-HC are expected to be low in unconditioned medium used for OGD, the difference in OGD neuronal survival between KO and WT cultures was surprising. It was hypothesized that despite low basal concentrations, toxic insults may increase extracellular 24S-HC concentration in medium during insult to levels that modulate NMDAR dependent damage. To test whether OGD increases 24S-HC concentration to levels necessary to exacerbate OGD neuronal injury, MEM medium was sampled immediately after the 2.5 hours OGD insult. It was found that 24S-HC concentration in OGD-challenged cultures was not different than control dishes (FIG. 2B and FIG. 2C). Moreover, absolute, bulk levels of 24S-HC in the MEM medium of both OGD and control cultures were very low compared with 24S-HC levels in the original conditioned medium (CM; FIG. 2B), as a result of the short conditioning period of the fresh, low-glucose MEM medium. Following insult, cells were returned to the original conditioned medium containing full glucose concentration. Levels of 24S-HC in this medium were measured 1 hour later and were not significantly elevated by the preceding OGD insult (FIG. 2B and FIG. 2C). Basal levels of 24S-HC in conditioned medium from mouse cultures were 14.06±2.80 ng/ml, or approximately 34.92 nM (FIG. 2B). This concentration is likely just-threshold for eliciting NMDAR potentiation (11) and thus could participate in exacerbating NMDAR-induced damage to explain the difference in toxicity between genotypes (FIG. 2A). Comparator 24S-HC concentrations from one KO culture were 0.49 ng/ml (˜1.22 nM) in control conditioned medium and 0.51 ng/ml (˜1.27 nM) in OGD conditioned medium. The low levels in KO culture conditioned medium verify that WT 24S-HC results from ongoing enzymatic synthesis. Overall, the results suggest that bulk 24S-HC levels could contribute to OGD damage in mouse cultures, and local levels of 24S-HC near sites of release may be even higher. However, the results do not support the idea that 24S-HC levels are elevated by insult.

### Example 3

### 25-HC is Partially Neuroprotective Against 24S-HC Exacerbated Damage and Against Damage in the Absence of 24S-HC

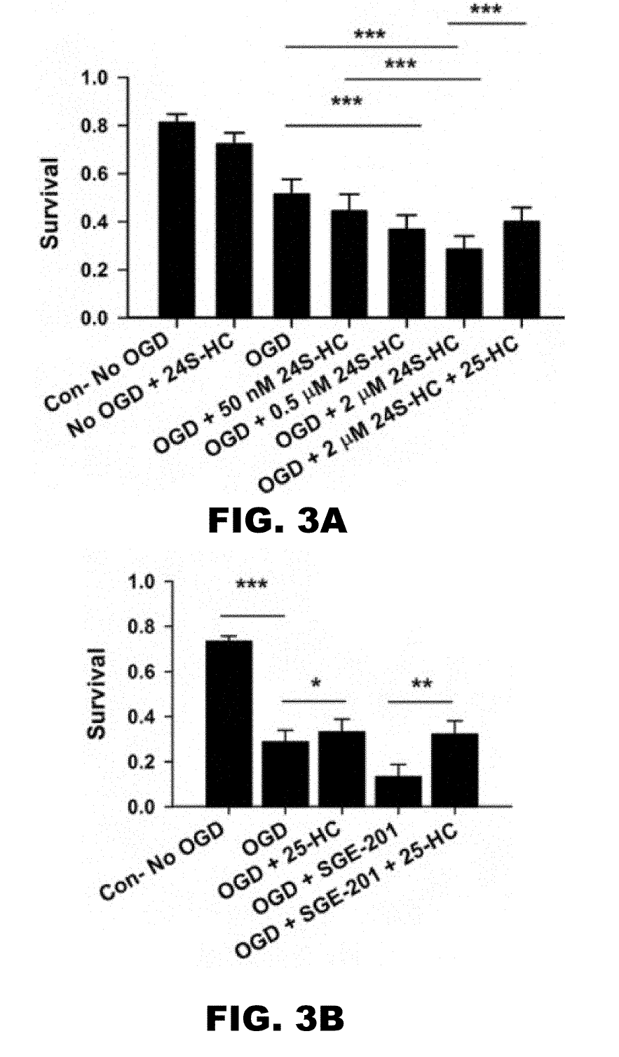

OGD-induced cell death in rat cultures depended on 24S-HC concentration over a range of 50 nM to 2 μM (FIG. 3A). These results suggest that local levels of 24S-HC produced in WT cells are not saturating. The oxysterol 25-HC non-competitively antagonizes 24S-HC effects on NMDARs. Here it was found that co-treatment with 10 μM 25-HC and 2 μM 24S-HC significantly alleviated OGD-induced cell death relative to that induced by 24S-HC and OGD alone (FIG. 3A). Similarly, 1 μM SGE-201, a 24S-HC analogue (11), exacerbated OGD injury, and this was alleviated by 10 μM 25-HC (FIG. 3B). Even without exogenous application of 24S-HC, OGD-induced cell death was alleviated by 10 μM 25-HC incubation during the OGD insult (FIG. 3B). This result could arise from antagonism of unappreciated local, endogenous 24S-HC activity. However, because measured bulk endogenous 24S-HC levels are low, it is also possible that, in additional to indirectly affecting NMDAR activity by antagonizing 24S-HC-induced potentiation of NMDARs, 25-HC may have NMDAR-independent neuroprotective effects against OGD damage.

### Example 4

### Evidence for NMDAR Independent Neuroprotective Effects of 25-HC

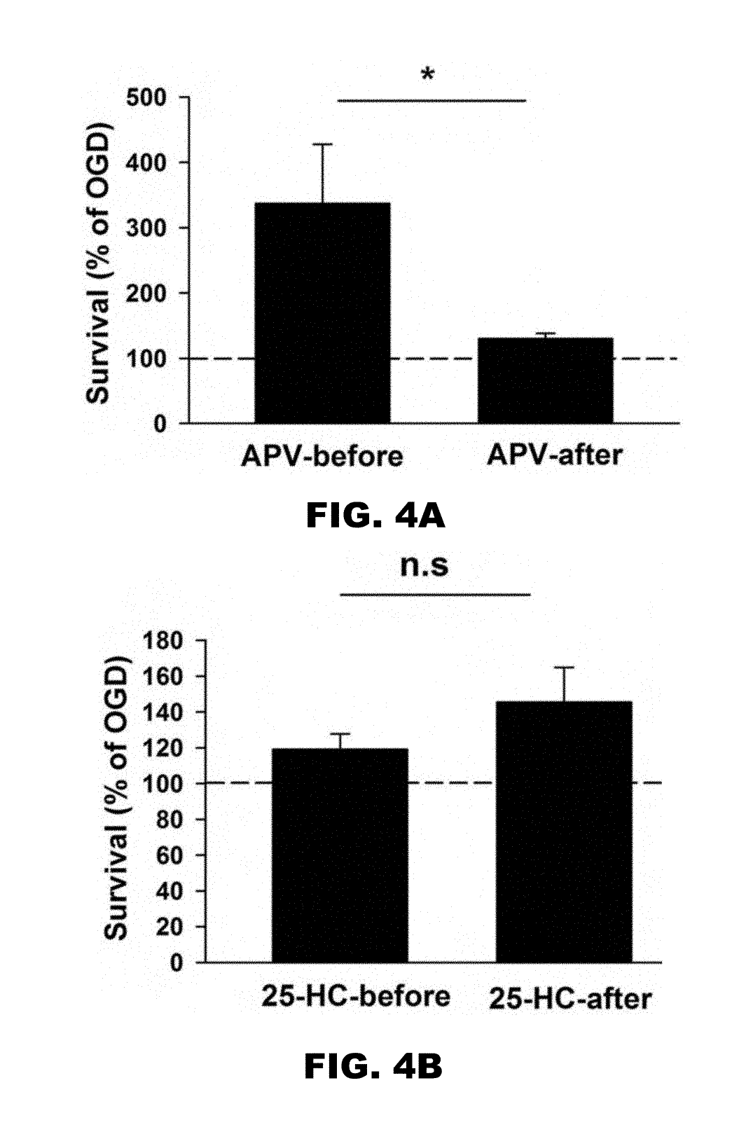

To test whether all of 25-HC's protective effects involve NMDARs, 25-HC was applied at different time points relative to the OGD insult, where the contribution of NMDARs is expected to vary. As a probe of NMDAR involvement at various stages during and after OGD, APV neuroprotection was tested in a separate group of cultures. As expected, APV was highly neuroprotective when administered before and during OGD. APV was much less effective when administered during the latent period following OGD insult, demonstrating the minor contribution of NMDAR activation in the latent period following OGD (FIG. 4A). If 25-HC acts through NMDARs, a corresponding decrease in effectiveness when 25-HC is administered following insult would be expected. By contrast, cultures treated with 25-HC before or after OGD insult exhibited comparable survival rates (FIG. 4B). Thus, despite the minor contribution of NMDARs following OGD, 25-HC neuroprotection was not altered, suggesting the possibility that OGD elicits a degree of NMDAR independent cell death, against which 25-HC is particularly neuroprotective.

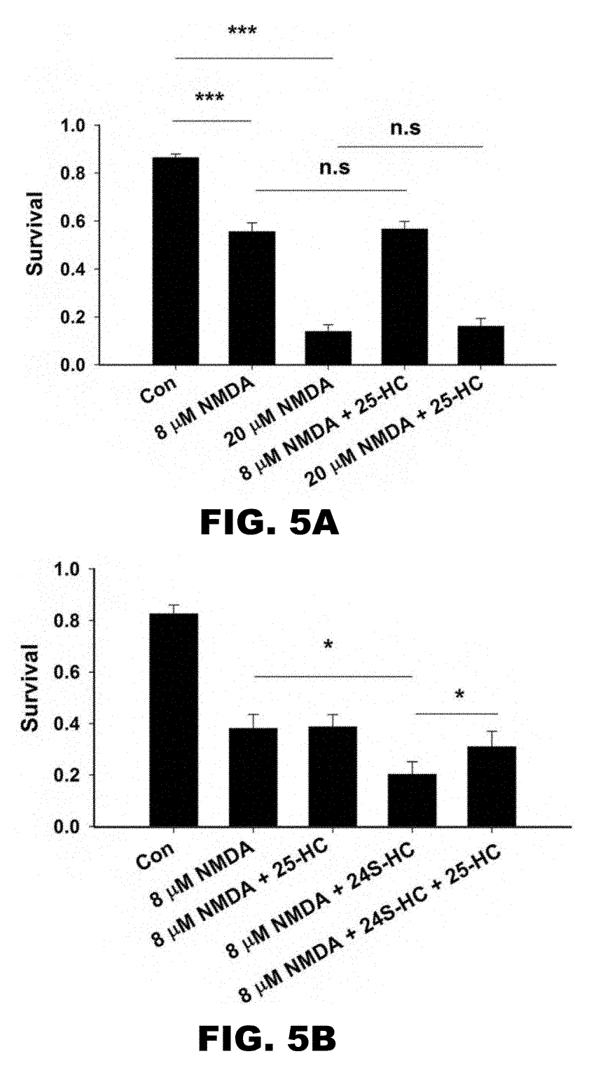

If the neuroprotective effects of 25-HC are entirely through antagonism of NMDAR function and endogenous, local 24S-HC, it would be expected that 25-HC should also reduce direct NMDA-induced cell death. NMDA was exogenously applied at either 8 μM or 20 μM to induce mild or severe toxicity (FIG. 5A). Under these conditions, 25-HC exhibited no neuroprotection, regardless of the severity of NMDA toxicity (FIG. 5A). However, co-treatment of 24S-HC exacerbated NMDA toxicity, and this exacerbation was significantly alleviated by 25-HC (FIG. 5B). These results suggest that 25-HC does not affect basal NMDAR activity by antagonizing local 24S-HC actions during excitotoxicity.

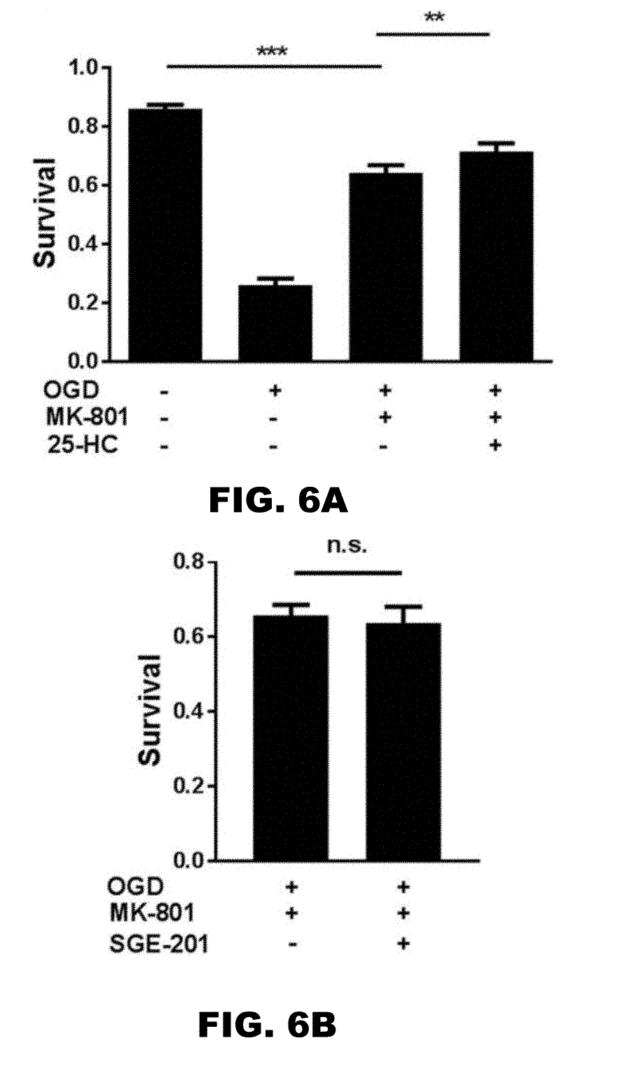

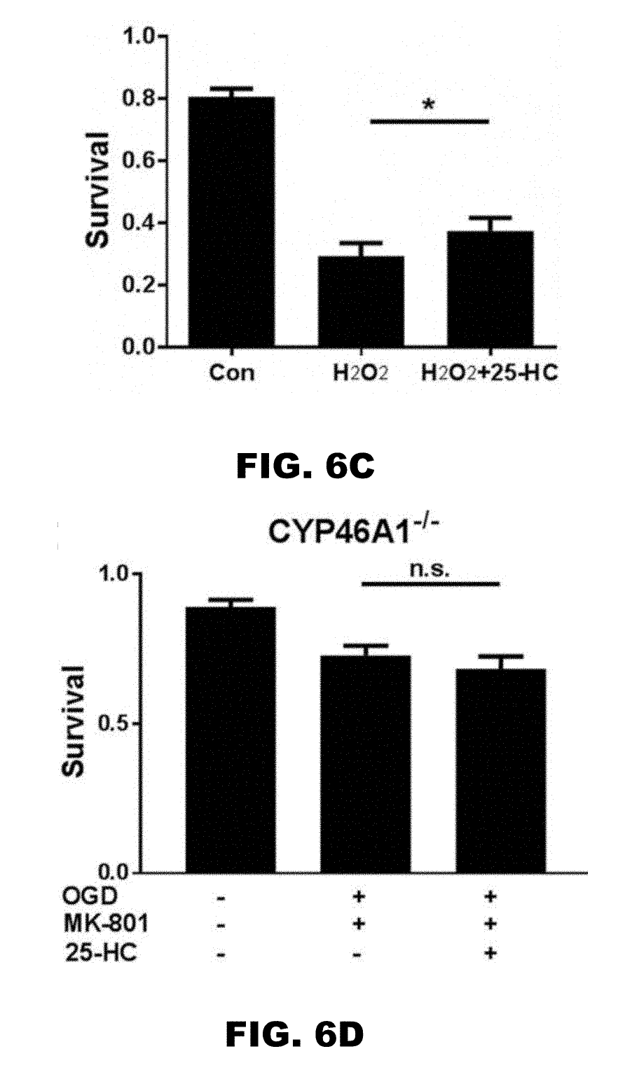

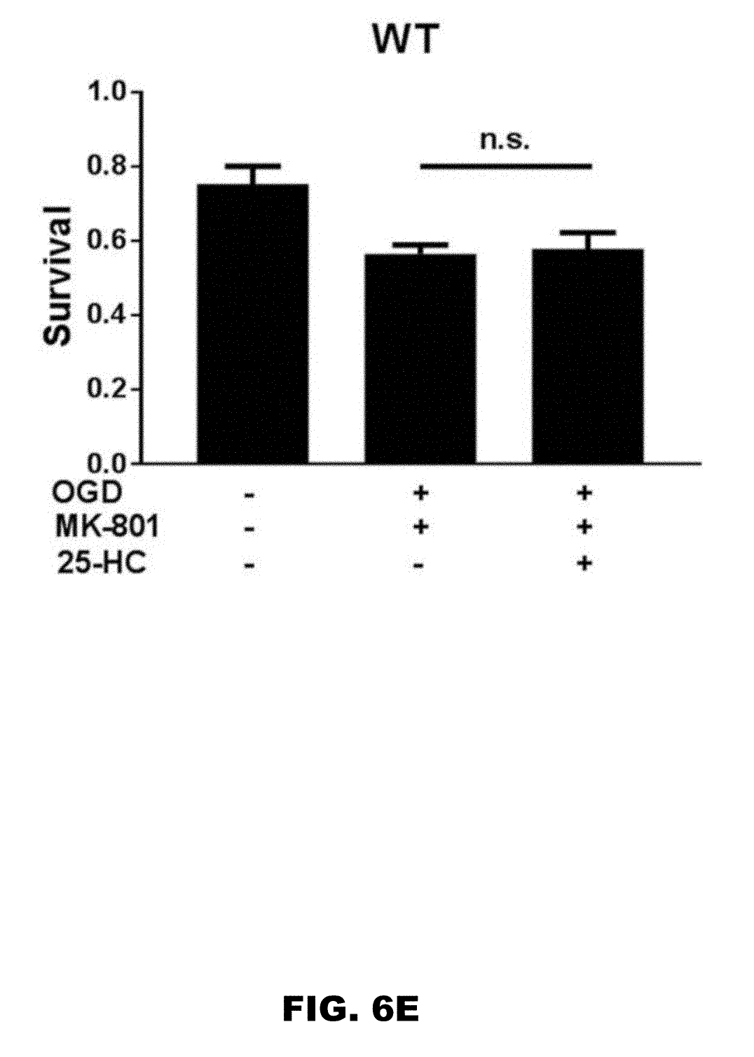

These results so far suggested that 25-HC may protect neurons during and after OGD, independent of NMDAR activity. As a final test, a more severe OGD insult was applied by increasing OGD exposure time from 2.5 hours to 3 hours to help emphasize any NMDAR-independent mechanisms of OGD-induced death. Under these conditions, cell death was only be partially rescued by NMDAR blockade with 20 μM MK-801, a non-competitive NMDAR channel blocker. To ensure that NMDARs were fully blocked under this condition, we co-treated cultures with 1 μM SGE-201. If unblocked NMDARs contributed to the residual death in the presence of MK-801, exacerbation of cell loss would be expected. In contrast to this expectation, 1 μM SGE-201 failed to exacerbate OGD toxicity in the presence of 20 μM MK-801 (FIG. 6B) (0.66±0.03 normalized survival for OGD+MK-801 and 0.64±0.04 normalized survival with SGE-201; n=9 cultures, P>0.05), suggesting complete inhibition of NMDAR activity. Nevertheless, 10 μM 25-HC yielded mild but significant neuroprotection in the presence of 20 μM MK-801 (FIG. 6A). This finding strongly supports the hypothesis that 25-HC is neuroprotective against OGD-induced cell death through a mechanism independent of NMDAR inhibition.

To further test the NMDAR-independence neuroprotective effect, a model of oxidative damage: H₂O₂ induced toxicity was used. In the presence of MK-801 to eliminate contributions of NMDARs, 25-HC had a similarly mild but consistent neuroprotective effect to that observed in OGD insult (FIG. 6C). However, NMDAR-independent neuroprotective effect did not extend to either WT or CYP46A1 knockout cultures (FIG. 6D and FIG. 6E).

### Discussion for the Examples.

These results extend the understanding of a class of NMDAR positive allosteric modulator. 24S-HC is the main cholesterol metabolite in brain and is responsible for brain cholesterol elimination as new cholesterol is synthesized (3). The results of the present study support the idea that 24S-HC may contribute to neuronal death in certain circumstances, in addition to serving as a biomarker of cell death. The results presented herein support the idea that in OGD, 24S-HC's exacerbation of damage is mainly through NMDARs. By contrast a less abundant oxysterol, 25-HC, acts as a neuroprotective agent through both NMDAR-dependent and NMDAR-independent mechanisms.

These results revealed that OGD-induced cell death exacerbated by 24S-HC can be prevented by the NMDAR antagonists APV (FIG. 1B) and MK-801 (FIG. 6), suggesting that 24S-HC exacerbated injury is NMDAR dependent. Consistent effects of 24S-HC were not observed when administered alone (FIG. 1B), and the primary effect of 24S-HC in the present work was an NMDAR dependent exacerbation of OGD-induced neuronal loss.

These studies included evaluation of the potential role of endogenous 24S-HC. Endogenous 24S-HC exacerbated OGD-induced cell death (FIG. 2). However, because OGD was performed in 24S-HC-free medium (FIG. 2B), it was posited that local 24S-HC levels, near sites of release, must be responsible for the effects of genetic overexpression and underexpression of CYP46A1. Local 24S-HC must exceed ˜50 nM (FIG. 3A), the exogenous concentration needed to potentiate NMDAR function. The results provided herein revealed no evidence that OGD increases 24S-HC release (FIG. 2C). Nevertheless, the possibility of local increases in 24S-HC concentration following OGD-induced NMDAR stimulation that were not reflected in bulk medium cannot be excluded.

25-HC antagonizes 24S-HC-mediated NMDAR potentiation. Interestingly, the results provided herein revealed neuroprotection by 25-HC against OGD-induced cell death, either in the presence or absence of exogenous 24S-HC application. The protective effect against exogenous 24S-HC exacerbated toxicity is NMDAR dependent (FIG. 3 and FIG. 5B). However, 25-HC appears to have another, small protective effect, revealed in the absence of 24S-HC or in the absence of NMDAR contributions, via an NMDAR-independent mechanism (FIG. 4, FIG. 5, and FIG. 6). This effect appears to account for most of the neuroprotective effect of 25-HC observed in the absence of exogenous NMDAR potentiator (FIG. 4, FIG. 5, and FIG. 6). The lack of detectable effect of 25-HC on the endogenous 24S-HC actions in OGD and NMDA toxicity (FIG. 4B and FIG. 5) could result from the low levels of 24S-HC present.

In summary, results from this study support the hypothesis that 24S-HC exacerbates NMDAR-dependent excitotoxicity induced by OGD—an ischemia-like challenge. In addition, 25-HC protects against OGD-induced cell death, even when administered following the insult. While 25-HC rescues NMDAR-dependent cell death exacerbated by 24S-HC, 25-HC exhibits NMDAR-independent neuroprotection against OGD-induced cell death. Our findings suggest that two oxysterols may both contribute to severity of damage and be palliative targets in ischemic stroke.

### Methods for the Examples.

Cell culture. All animal care and experimental procedures were consistent with National Institutes of Health guidelines and were approved by the Washington University Animal Studies Committee. Studies involving animals are reported in accordance with the ARRIVE guidelines for reporting experiments involving animals (12, 13). Rat or mouse hippocampal cultures were prepared from postnatal day 1 to 3, pups of both sexes (85% female) anaesthetized with isoflurane. Hippocampal slices (500 μm thickness) were digested with 1 mg⋅mL⁻¹ papain in oxygenated LeibovitzL-15 medium (Life Technologies, Gaithersburg, Md., USA). Tissue was mechanically triturated in modified Eagle's medium (Life Technologies) containing 5% horse serum, 5% FCS, 17 mM D glucose, 400 μM glutamine, 50 U⋅mL⁻¹ penicillin and 50 μg⋅mL⁻¹ streptomycin. Cells were seeded in modified Eagle's medium at a density of ˜650 cells per mm² (onto 25 mm cover glasses coated with 5 mg⋅mL⁻¹ collagen or 0.1 mg⋅mL⁻¹ poly-D-lysine with 1 mg⋅mL⁻¹ laminin). Cultures were incubated at 37° C. in a humidified chamber with 5% CO₂/95% air. Cytosine arabinoside (6.7 μM) was added 3-4 days after plating to inhibit glial proliferation. The following day, half of the culture medium was replaced with Neurobasal medium (Life Technologies) plus B27 supplement (Life Technologies).

Oxygen-glucose deprivation. Mass cultures (13-14 DIV) were exposed to oxygen-glucose deprivation (OGD), in which original medium containing 25 mM glucose was exchanged for fresh MEM medium with 2.5 mM glucose and exposed to a sealed chamber (Billups-Rothenberg, Del Mar, Calif., USA), humidified and saturated with 95% nitrogen and 5% CO₂ at 37° C., for 2.5 hours. The gas exchange followed the specifications of the chamber manufacturer (flow of 20 L⋅minute⁻¹ for 4 minutes to achieve 100% gas exchange). In some experiments, original medium was exchanged for fresh MEM medium containing the specified drugs immediately prior to OGD exposure. Controls were incubated in MEM medium without oxygen deprivation. Following OGD or control treatment, cells were returned to their original medium and incubated under standard culture conditions until the cell death assay (24 hours later). Hoechst 33342 (5 μM) was used to identify all nuclei and propidium iodide (PI, 3 μM) for 30 minutes to stain nuclei of cells with compromised membranes.

Chemicals. SGE-201 and SGE-108 were synthesized by previously published methods (11). D-APV and MK-801 were purchased from Tocris (Bristol, UK). 25-HC was purchased from Sigma (St. Louis, Mo.).

Metabolomics measurement of 24S-HC level in cell culture medium. 24-hydroxycholesterol in each medium (100 μL) sample was extracted with 400 μL of methanol. Deuterated 24-hydroxycholesterol-d₇ (10 ng) was added to each medium sample before extraction. Extracted 24-hydroxycholesterol and the internal standard were derivatized with N,N-dimethylglycinate (DMG) to increase the MS sensitivity. Oxysterol analysis was performed with a Shimadzu 20AD HPLC system, a LEAP PAL autosampler coupled to a triple quadrupole mass spectrometer (API 4000) operated in MRM mode. The positive ion ESI mode was used for detection of derivatized oxysterols. The study samples were injected in duplicate for data averaging. Data processing was conducted with Analyst 1.5.1 (Applied Biosystems). Quantification of 24S-hydroxycholesterol was determined by the deuterium labeled internal standard spiked in each sample. 24-hydroxycholesterol data were normalized as ng/mL for mediums.

Data analysis. Five 10× microscope fields were quantified per condition per experiment, yielding>100 total neurons for each condition. Ratios of healthy neurons were quantified as the fraction of PI-negative neuronal nuclei to total neuronal nuclei. Automated cell counting algorithms (ImageJ software, National Institutes of Health, Bethesda, Md., USA) were used for cell counts. Toxicity experiments were treated as a dependent sample design (32) in which sibling cultures plated in identical media and exposed to OGD at the same time were compared by repeated measures statistics. Results are shown as means±SEM. Comparative statistics were performed with Student's t-test or one-way repeated measures ANOVA where indicated.

### REFERENCES FOR THE EXAMPLES

1. Hardingham G E, Bading H. Synaptic versus extrasynaptic NMDA receptor signalling: implications for neurodegenerative disorders. Nat Rev Neurosci. 2010; 11(10):682-96.
2. Weilinger N L, Maslieieva V, Bialecki J, Sridharan S S, Tang P L, Thompson R J. Ionotropic receptors and ion channels in ischemic neuronal death and dysfunction. Acta Pharmacol Sin. 2013; 34(1):39-48.
3. Russell D W, Halford R W, Ramirez D M, Shah R, Kotti T. Cholesterol 24-hydroxylase: an enzyme of cholesterol turnover in the brain. Annu Rev Biochem. 2009; 78:1017-40.
4. Smith L L, Ray D R, Moody J A, Wells J D, Van Lier J E. 24-hydroxycholesterol levels in human brain. J Neurochem. 1972; 19(3):899-904.
5. Meljon A, Theofilopoulos S, Shackleton C H, Watson G L, Javitt N B, Knolker H J, et al. Analysis of bioactive oxysterols in newborn mouse brain by LC/MS. J Lipid Res. 2012; 53(11):2469-83.
6. Muir K W. Glutamate-based therapeutic approaches: clinical trials with NMDA antagonists. Curr Opin Pharmacol. 2006; 6(1):53-60.
7. Kaku D A, Goldberg M P, Choi D W. Antagonism of non-NMDA receptors augments the neuroprotective effect of NMDA receptor blockade in cortical cultures subjected to prolonged deprivation of oxygen and glucose. Brain Res. 1991; 554(1-2):344-7.
8. Emnett C M, Eisenman L N, Taylor A M, Izumi Y, Zorumski C F, Mennerick S. Indistinguishable synaptic pharmacodynamics of the N-methyl-D-aspartate receptor channel blockers memantine and ketamine. Mol Pharmacol. 2013; 84(6):935-47.
9. Rothman S M, Thurston J H, Hauhart R E, Clark G D, Solomon J S. Ketamine protects hippocampal neurons from anoxia in vitro. Neuroscience. 1987; 21(3):673-8.
10. Goldberg M P, Weiss J H, Pham P C, Choi D W. N-methyl-D-aspartate receptors mediate hypoxic neuronal injury in cortical culture. J Pharmacol Exp Ther. 1987; 243(2):784-91.
11. Paul S M, Doherty J J, Robichaud A J, Belfort G M, Chow B Y, Hammond R S, et al. The major brain cholesterol metabolite 24(S)-hydroxycholesterol is a potent allosteric modulator of N-methyl-D-aspartate receptors. J Neurosci. 2013; 33(44):17290-300
12. Kilkenny C, Browne W J, Cuthill I C, Emerson M, Altman D G. Improving bioscience research reporting: the ARRIVE guidelines for reporting animal research. PLoS Biol. 2010; 8(6):e1000412.
13. Suchanek M, Hynynen R, Wohlfahrt G, Lehto M, Johansson M, Saarinen H, et al. The mammalian oxysterol-binding protein-related proteins (ORPs) bind 25-hydroxycholesterol in an evolutionarily conserved pocket. Biochem J. 2007; 405(3):473-80.

1. A method of decreasing neuronal cell death, the method comprising administering a composition comprising 25-hydroxycholesterol (25-HC), a 25-HC derivative, or a 25-HC prodrug.

2. The method of claim 1, wherein the neuronal cell death is due to ischemia.

3. The method of claim 1, wherein the neuronal cell death is due to stroke.

4. The method of claim 1, wherein the composition is administered following the onset of neuronal cell death.

5. The method of claim 1, wherein the composition comprises from about 1 μM to about 100 μM of 25-hydroxycholesterol (25-HC), a 25-HC derivative, or a 25-HC prodrug.

6. The method of claim 1, wherein the composition comprises from about 1 μM to about 50 μM of 25-hydroxycholesterol (25-HC), a 25-HC derivative, or a 25-HC prodrug.

7. The method of claim 1, wherein the composition comprises from about 5 μM to about 25 μM of 25-hydroxycholesterol (25-HC), a 25-HC derivative, or a 25-HC prodrug.

8. The method of claim 1, wherein the composition comprises 25-hydroxycholesterol.

9. The method of claim 8, wherein the administration step is performed by administering to a subject in need thereof.

10. The method of claim 9, wherein the subject is having or is suspected of having an ischemia.

11. The method of claim 9, wherein the subject is having or is suspected of having a stroke.

12. A method of treating or preventing stroke, the method comprising administering a composition comprising 25-hydroxycholesterol (25-HC), a 25-HC derivative, or a 25-HC prodrug.

13. The method of claim 12, wherein the stroke is ischemic stroke.

14. The method of claim 12, wherein the stroke is a transient ischemic attack.

15. The method of claim 12, wherein the composition comprises from about 1 μM to about 50 μM of 25-hydroxycholesterol (25-HC), a 25-HC derivative, or a 25-HC prodrug.

16. The method of claim 12, wherein the composition comprises from about 5 μM to about 25 μM of 25-hydroxycholesterol (25-HC), a 25-HC derivative, or a 25-HC prodrug.

17. The method of claim 12, wherein the composition comprises 25-hydroxycholesterol.

18. The method of claim 12, wherein the administration step is performed by administering to a subject in need thereof.

19. The method of claim 18, wherein the subject is having or is suspected of having an ischemia.

20. The method of claim 18, wherein the subject is having or is suspected of having a stroke.
